(19) United States
(12) Patent Application Publication
(10) Pub. No.: US 2018/0104015 A1
(43) Pub. Date: Apr. 19, 2018

(54) SANITATION STATUS INDICATOR FOR MEDICAL EQUIPMENT

(71) Applicant: IMD, Austin, TX (US)

(72) Inventors: Carey Bryant, Hernando, MS (US); Thomas Watts, Round Rock, TX (US)

(21) Appl. No.: 15/297,036

(22) Filed: Oct. 18, 2016

(51) Int. Cl. A61B 50/13; A61B 50/15; A61B 50/18; A61L 2/26; G09F 3/20

(52) U.S. Cl. CPC A61B 50/13; A61B 50/15; A61B 50/18; A61B 2090/081; G09F 3/208; G09F 3/20; A61L 2/26

(57) ABSTRACT

An apparatus for indicating a sanitation status of a portable surgical cart or medical tool supported by the portable surgical cart is disclosed herein. The apparatus comprises at least one indicium that visually indicates a sanitation status of the portable surgical cart. The sanitation status is one of at least sterilized or contaminated. The apparatus is configured to be coupled to the portable surgical cart.

### FIELD

The present application relates generally to medical devices, and more particularly to providing a visual indication of the sanitary condition of medical devices.

### BACKGROUND

During surgery, and other medical processes, medical practitioners often use a variety of medical tools to perform various procedures on a patient. For example, one or more scalpels may be used to make an incision, one or more retractors may be used to hold open the incision, and one or more scopes, probes, cutters, graspers, etc. may be used to operate on the patient. Often, sterile medical tools are arranged on a portable cart or tray in preparation for surgery. Then, after a medical tool has been used during surgery, the item should either be safely disposed of, as a biohazardous material, or the item should be properly cleaned and sterilized for reuse.

Disposable and non-sterile reusable medical tools, before being cleaned and sterilized, are often placed back on the same portable cart or tray used to deliver the sterile medical tools for the surgery. Once the surgery has been performed, a practitioner may take the portable cart, upon which the non-sterile medical tools are placed, and dispose of the disposable medical tools, as well as sterilize (e.g., clean) the reusable tools and the portable cart for future use. However, for various reasons, such as operator negligence, inattention, or workload, non-sterile portable carts, and medical tools on the carts, may not be immediately sanitized after a surgery, and thus the sanitation status of the portable cart and medical tools may not be easily identified. Because the sanitation status of the surgical cart and medical tools may not be clear from mere observation, practitioners may inadvertently use a non-sterilized surgical cart to support non-sterilized medical tools and/or sterilized medical tools for surgery, thus resulting in health risks. Alternatively, because the sanitation status of the surgical cart and medical tools may not be clear from mere observation, practitioners may re-sterilize previously sterilized surgical carts and/or medical tools, resulting in wasted time and resources.

### SUMMARY

The subject matter of the present application has been developed in response to the present state of the art, and in particular, in response to the shortcomings of sterilization practices associated with reusable surgical carts, medical tools, and other medical instruments, that have not yet been fully solved by currently available techniques. Accordingly, the subject matter of the present application has been developed to provide an apparatus and associated method that overcome at least some of the above-discussed shortcomings of currently available techniques.

An apparatus for indicating a sanitation status of a portable surgical cart or medical tool supported by the portable surgical cart is disclosed herein. The apparatus comprises at least one indicium that visually indicates a sanitation status of the portable surgical cart. The sanitation status is one of at least sterilized or contaminated. The apparatus is configured to be coupled to the portable surgical cart. The preceding subject matter of this paragraph characterizes example 1 of the present disclosure.

The apparatus comprises at least two indicium each visually indicating one of at least sterilized or contaminated. The preceding subject matter of this paragraph characterizes example 2 of the present disclosure, wherein example 2 also includes the subject matter according to example 1, above.

The apparatus further comprises a cover that is movable relative to at least the two indicium between a first position, covering only one of at least the two indicium, and a second position, covering another of at least the two indicium. The preceding subject matter of this paragraph characterizes example 3 of the present disclosure, wherein example 3 also includes the subject matter according to example 2, above.

The cover is slidable between the first position and the second position. The preceding subject matter of this paragraph characterizes example 4 of the present disclosure, wherein example 4 also includes the subject matter according to example 3, above.

The apparatus further comprises a base and a channel defined by the base, and wherein the cover is positioned within and slides along the channel. The preceding subject matter of this paragraph characterizes example 5 of the present disclosure, wherein example 5 also includes the subject matter according to example 4, above.

The cover is rotatable between the first position and the second position. The preceding subject matter of this paragraph characterizes example 6 of the present disclosure, wherein example 6 also includes the subject matter according to example 3, above.

The apparatus further comprises a base and a dial, the dial being rotatable relative to the base and including at least the two indicium thereon. The cover is non-movably coupled to the base such that the dial is interposed between the base and the cover. The preceding subject matter of this paragraph characterizes example 7 of the present disclosure, wherein example 7 also includes the subject matter according to example 6, above.

The cover includes a window sized to allow visual access therethrough to only one of at least the two indicium. The preceding subject matter of this paragraph characterizes example 8 of the present disclosure, wherein example 8 also includes the subject matter according to example 7, above.

The apparatus further comprises a detent mechanism that releasably retains the dial in each of the first position and the second position. The preceding subject matter of this paragraph characterizes example 9 of the present disclosure, wherein example 9 also includes the subject matter according to example 7, above.

The dial comprises a knob and the cover comprises a central aperture through which the knob of the dial extends. The preceding subject matter of this paragraph characterizes example 10 of the present disclosure, wherein example 10 also includes the subject matter according to example 7, above.

The apparatus further comprises a locking mechanism selectively operable to prevent movement of the cover between the first position and the second position. The preceding subject matter of this paragraph characterizes example 11 of the present disclosure, wherein example 11 also includes the subject matter according to any one of examples 3-10, above.

The apparatus further comprises an electronic display that is switchable between displaying a sanitation status, corresponding with sterilized, and a second sanitation status, corresponding with contaminated. The preceding subject matter of this paragraph characterizes example 12 of the present disclosure, wherein example 12 also includes the subject matter according to any one of examples 1 to 2, above.

The apparatus further comprises a flip-chart mechanism. The preceding subject matter of this paragraph characterizes example 13 of the present disclosure, wherein example 13 also includes the subject matter according to example 1, above.

The apparatus further comprises at least one liquid drainage port. The preceding subject matter of this paragraph characterizes example 14 of the present disclosure, wherein example 14 also includes the subject matter according to any one of examples 1 to 13, above.

The apparatus further comprises opposing, angled, or orthogonal sides each including at least the one indicium. The preceding subject matter of this paragraph characterizes example 15 of the present disclosure, wherein example 15 also includes the subject matter according to any one of examples 1 to 14, above.

A system is disclosed herein. The system comprises a portable surgical cart for holding medical tools. The system also comprises an apparatus coupled to the portable surgical cart. The apparatus comprises at least one indicium that visually indicates a sanitation status of the portable surgical cart, wherein the sanitation status is one of at least sterilized or contaminated. The preceding subject matter of this paragraph characterizes example 16 of the present disclosure.

The apparatus is permanently mounted to the portable surgical cart. The preceding subject matter of this paragraph characterizes example 17 of the present disclosure, wherein example 17 also includes the subject matter according to example 16, above.

The apparatus is selectively releasably mounted to the portable surgical cart. The preceding subject matter of this paragraph characterizes example 18 of the present disclosure, wherein example 18 also includes the subject matter according to example 16, above.

The portable surgical cart comprises a plurality of shelves. The system further comprises a plurality of apparatuses each coupled to a respective one of the plurality of shelves. The preceding subject matter of this paragraph characterizes example 19 of the present disclosure, wherein example 19 also includes the subject matter according to any one of examples 16 to 18, above.

A method is disclosed herein. The method comprises sterilizing a portable surgical cart after the portable surgical cart has been contaminated from a first surgery. The method also comprises, after sterilizing the portable surgical cart, switching a sanitation status of an apparatus that is coupled to the portable surgical cart from a contaminated sanitation status to a sterilized sanitation status. The method additionally comprises using the portable surgical cart, with the sterilized sanitation status, in a second surgery. The method further comprises, after or during use of the surgical cart in the second surgery, switching the sanitation status of the apparatus from the sterilized sanitation status to the contaminated sterilized sanitation status. The preceding subject matter of this paragraph characterizes example 20 of the present disclosure.

The described features, structures, advantages, and/or characteristics of the subject matter of the present disclosure may be combined in any suitable manner in one or more embodiments and/or implementations. In the following description, numerous specific details are provided to impart a thorough understanding of embodiments of the subject matter of the present disclosure. One skilled in the relevant art will recognize that the subject matter of the present disclosure may be practiced without one or more of the specific features, details, components, materials, and/or methods of a particular embodiment or implementation. In other instances, additional features and advantages may be recognized in certain embodiments and/or implementations that may not be present in all embodiments or implementations. Further, in some instances, well-known structures, materials, or operations are not shown or described in detail to avoid obscuring aspects of the subject matter of the present disclosure. The features and advantages of the subject matter of the present disclosure will become more fully apparent from the following description and appended claims, or may be learned by the practice of the subject matter as set forth hereinafter.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention, and are not therefore to be considered to be limiting of its scope, the invention will be described and explained with additional specificity and detail through the use of the accompanying drawings, in which:

FIG. 1 is a perspective view of a system that includes a surgical cart and an apparatus for indicating the sanitation status of the surgical cart, according to one or more embodiments of the present disclosure;

FIG. 2 is a perspective view of a system that includes a surgical cart and apparatuses each indicating the sanitation status of respective portions of the surgical cart, according to one or more embodiments of the present disclosure;

FIG. 3 is a perspective view of a system that includes a surgical cart and an apparatus for indicating the sanitation status of the surgical cart, according to one or more embodiments of the present disclosure;

FIG. 4 is a perspective view of a system that includes a surgical cart and an apparatus for indicating the sanitation status of the surgical cart, according to one or more embodiments of the present disclosure;

FIG. 5 is a perspective view of a system that includes a surgical cart and an apparatus for indicating the sanitation status of the surgical cart, according to one or more embodiments of the present disclosure;

FIG. 6A is a front view of an apparatus for indicating the sanitation status of a surgical cart or medical tools, shown indicating a first sanitization condition, according to one or more embodiments of the present disclosure;

FIG. 6B is a front view of the apparatus of FIG. 6B, shown indicating a second sanitization condition, according to one or more embodiments of the present disclosure;

FIG. 7 is a perspective view of an apparatus for indicating the sanitation status of a surgical cart or medical tools, according to one or more embodiments of the present disclosure;

FIG. 8 is a front view of the apparatus of FIG. 7, according to one or more embodiments of the present disclosure;

FIG. 9 is a back view of the apparatus of FIG. 7, according to one or more embodiments of the present disclosure;

FIG. 10 is a perspective view of an apparatus for indicating the sanitation status of a surgical cart or medical tools, according to one or more embodiments of the present disclosure;

FIG. 11 is a perspective view of the apparatus of FIG. 10, shown with a cover removed, according to one or more embodiments of the present disclosure;

FIG. 12 is an exploded perspective view of the apparatus of FIG. 10, according to one or more embodiments of the present disclosure;

FIG. 13 is a cross-sectional side elevation view of the apparatus of FIG. 10, according to one or more embodiments of the present disclosure; and

FIG. 14 is a schematic flowchart diagram illustrating a method of indicating the sanitation status of a surgical cart or medical tools, according to one or more embodiments of the present disclosure.

### DETAILED DESCRIPTION

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

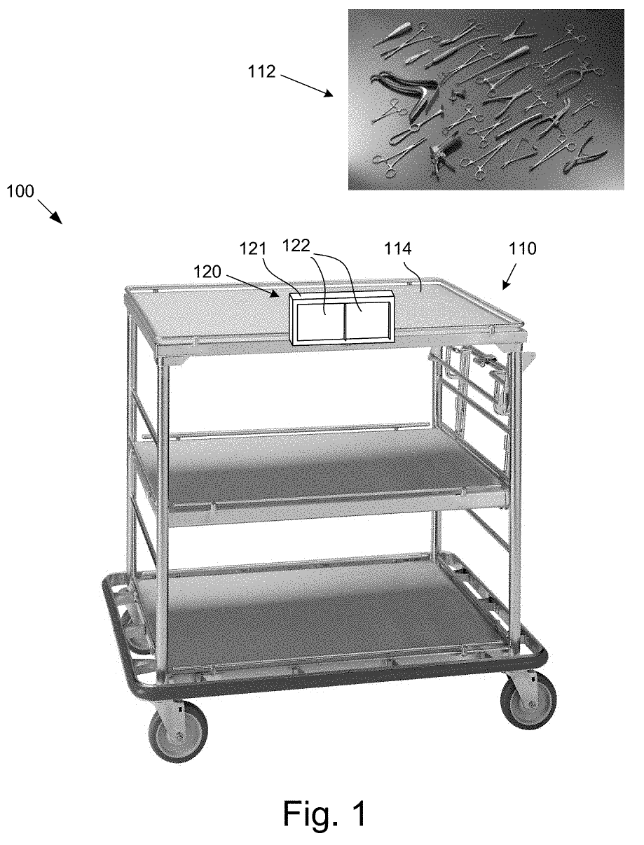

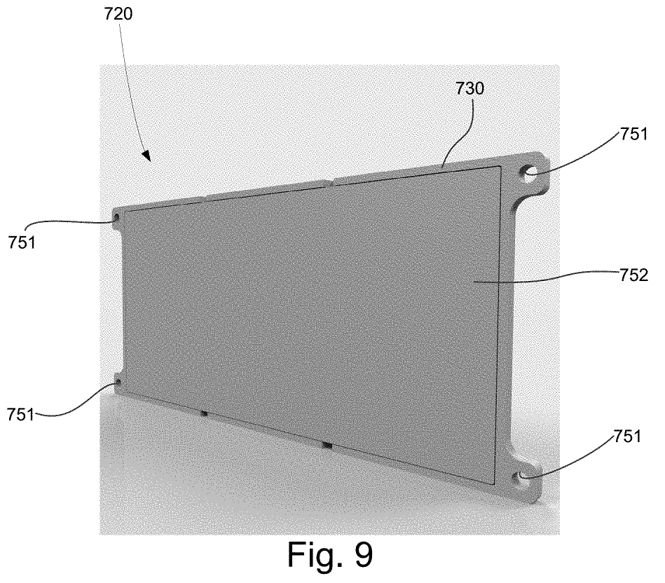

Illustrated in FIGS. 1-11 are several representative embodiments of an apparatus for indicating a sanitation status of a surgical cart, medical tools, or other medical instrument. Also described herein is one or more methods of using the apparatus to help indicate the sanitation status of medical instruments. As described herein, the apparatus provides several significant advantages and benefits over conventional practices. However, the recited advantages are not meant to be limiting in any way, as one skilled in the art will appreciate that other advantages may also be realized upon practicing the present disclosure.

According to FIG. 1, an apparatus 120 for indicating the sanitation status of one or more medical instruments forms part of a system 100. The system 100 includes a medical instrument, such as a portable surgical cart 110, and the apparatus 120, which is secured to or otherwise in close proximity with the medical instrument. The portable surgical cart 110 is configured to hold any of various medical tools 112, which can be reusable or disposable medical tools. For example, the medical tools 112 may include forceps, clamps, retractors, distractors, scalpels, drill bits, dilators, suction tips, surgical staplers, injection needles, drills, saws, powered tools, scopes, probes, rulers, calipers, etc. In one embodiment, the medical tools 112 can be held on one or more shelves 114 of the portable surgical cart 110. Prior to surgery, the medical tools 112, in a sanitized condition or state, are placed on the surgical cart 110, also in a sanitized condition or state.

As described above, after one or more of the medical tools 112 has been used in a surgery or other medical procedure, the medical tools 112, now in a non-sanitized state, may be placed back down on the surgical cart 110, thus changing the condition of the surgical cart 110 to a non-sanitized condition. Additionally, or alternatively, the surgical cart 110 may be contaminated during surgery in other ways, such as through contact with germs, fluids, tissue and the like, associated with surgical procedures. Because of the contamination of the surgical cart 110 and medical tools 112 during surgery, the surgical cart 110 and the medical tools 112 must be cleaned and sterilized before the surgical cart 110 and the medical tools 112 are used again in a subsequent surgery or other medical procedure. Because it may be difficult to know the sanitation status of the surgical cart 110 and/or the medical tools 112 by mere observation, the apparatus 120 can be used to visually indicate to medical personnel the sanitation status of the surgical cart 110 and/or the medical tools 112 on the surgical cart 110.

The apparatus 120 has a switchable indicator 122 that can be changed to display different characters corresponding with different sanitation statuses of the surgical cart 110 and/or medical tools 112 on the surgical cart 110. In this manner, the apparatus 120 helps medical personnel track the sterilization condition of the surgical cart 110 and/or medical tools 112 on the surgical cart 110 before, after, and during surgery.

The apparatus 120 is secured to the surgical cart 110 using any of various methods. In one embodiment, the apparatus 120 is co-formed with the portable surgical cart 110 so as to be integrated (e.g., form a one-piece monolithic construction) with the surgical cart 110. In other embodiments, the apparatus 120 is separately formed and attached to the portable surgical cart 110. In one implementation, the apparatus 120 is attached to the portable surgical cart 110 by permanently coupling the apparatus 120 to the portable surgical cart 110, such as via welding, bonding, adhesion, and the like. In another implementation, the apparatus 120 is selectively releasably attached to the portable surgical cart 110, such as via bolts, fasteners, hook and loop couplings, quick-release couplings, clamps, and the like. The portable surgical cart 110 and the apparatus 120 are made of materials that can be repeatedly sanitized. In some implementations, the portable surgical cart 110 and the apparatus 120 are made of the same material, and in other implementations, the portable surgical cart 110 and the apparatus 120 are made of different materials. For example, in one implementation, the surgical cart 110 is made from a metallic material, such as stainless steel, and the apparatus 120 is made from a hardened plastic or polymeric material. Alternatively, in another implementation, the apparatus 120 is made from a metallic material, such as stainless steel.

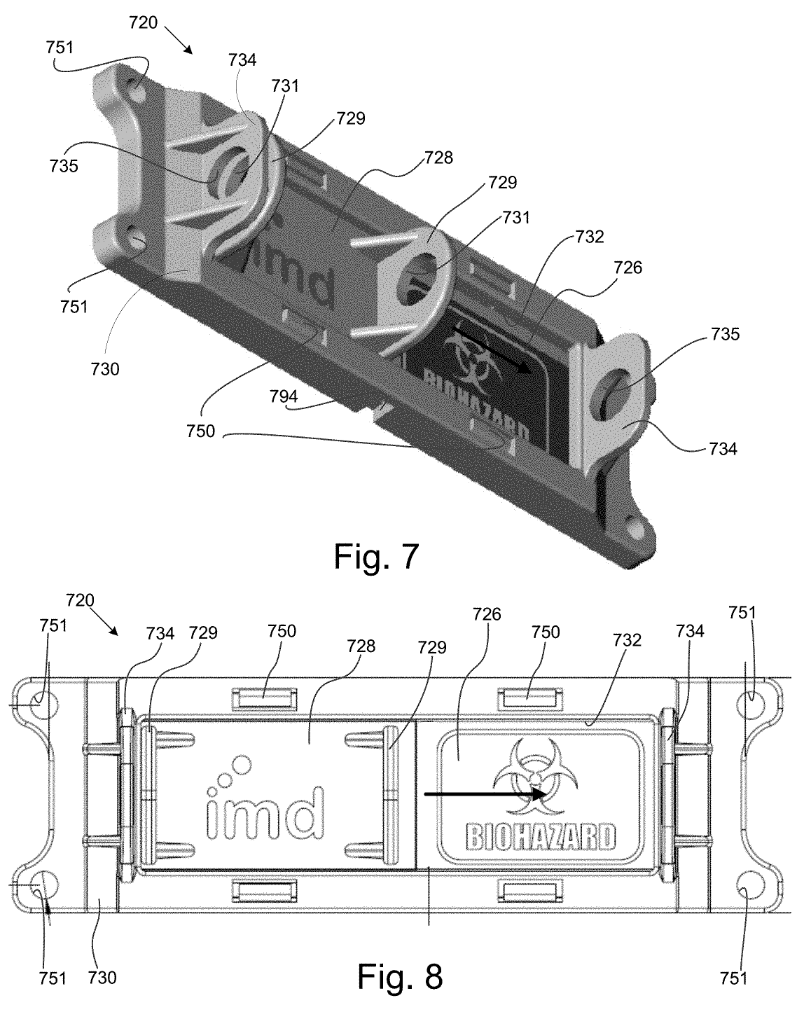

The apparatus 120 includes a housing 121 that at least partially houses the switchable indicator 122. The housing 121 may also include one or more apertures or liquid drainage ports (see, e.g., liquid drainage port 794 in FIG. 7) that allow sterilization fluid or other fluid to drain from the apparatus 120. Generally, the switchable indicator 122 includes at least one indicium indicating a sanitation status. In some embodiments, the switchable indicator 122 includes indicia indicating at least two sanitation statuses where one indicium indicating a first sanitation status (e.g., non-sanitized or hazardous materials) is hidden, or visually obstructed, while another indicium indicating a second sanitation status (e.g., sanitized or clean) is visually accessible. When the sanitation status of the surgical cart 110 and/or medical tools 112 changes (e.g., during or following surgery), the switchable indicator 122 can be switched to make visually accessible the previously hidden indicium indicating the first sanitation status and obstruct the previously accessible indicium indicating the first sanitation status.

The term “sanitation status” is defined herein as a designation of a sterilization condition of the surgical cart 110 and/or medical tools 112. In other words, the apparatus 120 is selectively operable to indicate the cleanliness of the portable surgical cart 110 and/or medical tools 112. Further description and details relating to various embodiments of the portable surgical cart 110 and the apparatus 120 are included below with reference to the remaining figures. Throughout the present disclosure, unless otherwise indicated, like reference numbers refer to like elements.

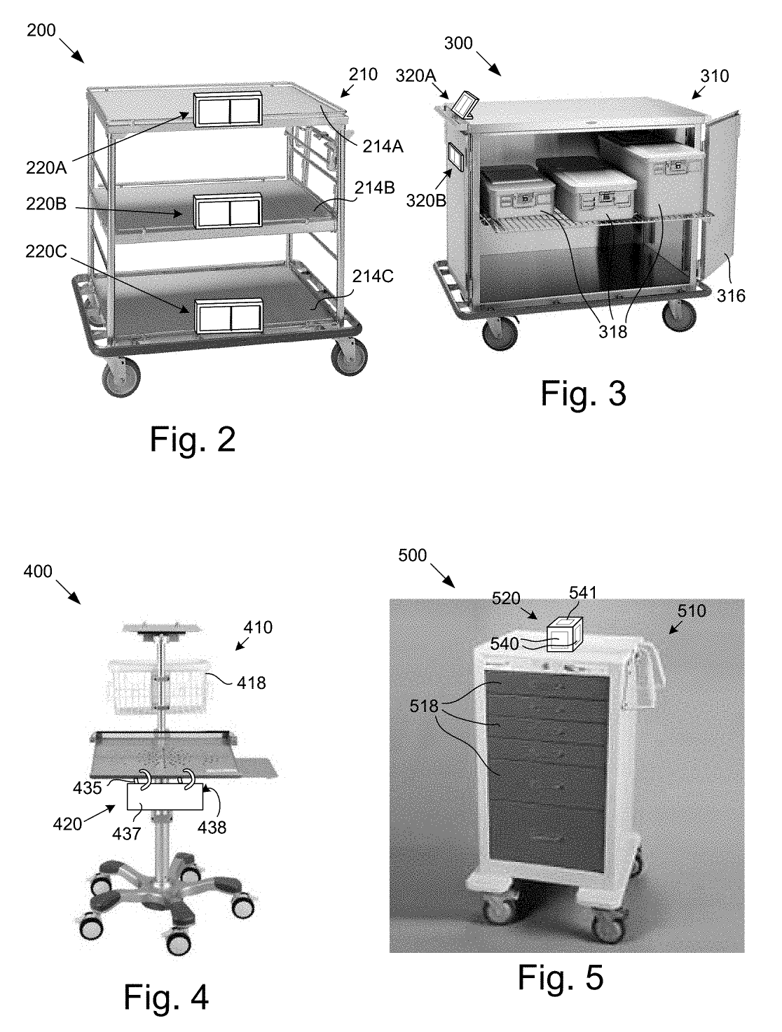

According to another embodiment, as shown in FIG. 2, a system 200 includes multiple apparatuses 220A-C each secured to, or positioned proximate, a respective one of multiple shelves 214A-C of a portable surgical cart 210. In one embodiment, the shelves 214A-C are removably detachable from and/or repositionable within the portable surgical cart 210. In another embodiment, the shelves 214A-C are retractable and extendable. In yet another embodiment, the shelves 214A-C are non-movably and non-adjustably fixed relative to the portable surgical cart 210 and/or form a unitary one-piece structure with the portable surgical cart 210. Each of the apparatuses 220A-C indicates the sanitation status of a respective one of the shelves 214A-C, or the sanitation status of the medical tools 112 on a respective one of the shelves 214A-C. However, as shown in FIG. 1, although a portable surgical cart 110 may include multiple shelves, the system may include only one apparatus that indicates a sanitation status of all the shelves of the portable surgical cart 110.

FIG. 3 shows another embodiment of a system 300 that includes a portable surgical cart 310 with one or more doors 316 providing access to an interior chamber of the portable surgical cart 310. Furthermore, the portable surgical cart 310 may include one or more containers 318 within which reusable surgical equipment may be stored. In one embodiment, at least one apparatus for indicating a sanitation status of the portable surgical cart 310 and/or medical tools stored in or on the portable surgical cart 310 is mounted to an exterior of the portable surgical cart 310. For example, an apparatus 320A is mounted to a top surface of the portable surgical cart 310. The apparatus 320A is angled or otherwise oriented to improve the visibility of the apparatus 320A. In another example, an apparatus 320B is mounted (e.g., welded) to a side surface of the portable surgical cart 310. The system 300 may include one or both of the apparatuses 320A, 320B depicted in FIG. 3.

Referring now to FIG. 4, and according to another embodiment, a system 400 includes a portable surgical cart 410 and an apparatus 420, for indicating a sanitation status of the portable surgical cart 310 and/or medical tools stored in or on the portable surgical cart 310, coupled to the portable surgical cart 310. The apparatus 420 has a flip-chart design with at least one cover 438 or panel that moves or flips along rings 435 or other fasteners. More specifically, the cover 438 has a first side 437 and an opposing second side where each side includes one of two different indicia corresponding with two different sanitation statuses. A practitioner may flip the cover 438 to display either the first side 437 or the second side switch according to a known sanitation status of the portable surgical cart 310 and/or medical tools associated with the portable surgical cart 310. In one embodiment, the rings 435 of the apparatus 420 facilitate the coupling between the apparatus 420 and the portable surgical cart 410. The portable surgical cart 410 may include baskets 418 or other similar containers for holding and storing reusable or disposable medical tools.

As shown in FIG. 5, a system 500, according to yet another embodiment, includes a portable surgical cart 510 and an apparatus 520, for indicating a sanitation status of the portable surgical cart 510 and/or medical tools stored in or on the portable surgical cart 510, coupled to the portable surgical cart 510. The portable surgical cart 510 includes a plurality of drawers 518 for storing various types of reusable and/or disposable surgical equipment. In one implementation, the apparatus 520 freely rests on the portable surgical cart 510 and has multiple opposing, angled, or orthogonal sides 540, 541. Each side 540, 541 includes the same or different indicia corresponding with at least one sanitation status. In one implementation, at least two of the sides 540, 541 include the same indicia such that personnel can view the same indicia from multiple angles relative to the apparatus 520 (e.g., viewable 360° around the portable surgical cart 510). In another implementation, the side 541 of the apparatus 520 is a top side that allows personnel to view the indicia on the side 541, and ascertain a sanitation status of the portable surgical cart 510 and/or medical tools, from above the portable surgical cart 510. For example, as depicted, the apparatus 520 is cubicle in shape and has four lateral sides 540 and two top and bottom sides 541. In another example, the apparatus 520 is spherical or shaped like a pyramid to allow users to view the display unit from various positions around the surgical cart 510.

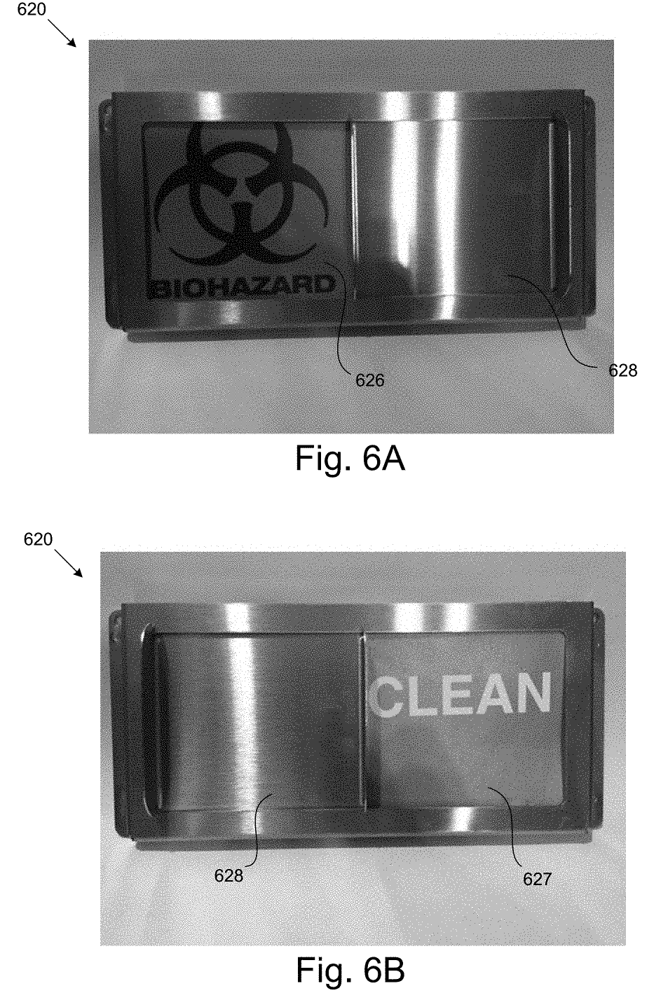

Referring to FIGS. 6A and 6B, according to one embodiment, an apparatus 620 that is switchable between visually indicating a first indicium 626 or visually indicating a second indicium 627. As depicted, the first indicium 626 indicates a non-sanitized or biohazard condition and the second indicium 627 indicates a sanitized or clean condition. Switching between visually indicating the first indicium 626 or the second indicium 627 is accomplished via a movable cover 628. The movable cover 628 is made of an opaque or non-transparent material such that when covering an indicium, the indicium is visually obstructed or visually inaccessible. In one implementation, the movable cover 628 is slidable relative to the first and second indicia 626, 627 to cover either the first or second indicia 626, 627, and thus make visually accessible the other of the first or second indicia 626, 627.

As used herein, the term “indicium” refers to any numbers, letters, phrases, pictures, colors, symbols, images, logos, etc. that indicate and convey to a user the sanitation status of a surgical cart and/or medial tools. As shown in FIGS. 6A and 6B, in one particular implementation, the first indicium 626 includes a biohazard symbol and the second indicium 627 includes the word “clean.” In other implementations, the apparatuses described herein incorporate lights, colors, sounds, etc. to clearly designate, indicate, and convey a sanitation status to personnel.

In one embodiment, an apparatus, for indicating a sanitation status of a portable surgical cart and/or medical tools stored in or on the portable surgical cart, includes a banner that wraps at least partially around a base of the apparatus and that is detachable/swappable relative to the base. For example, a switchable banner may be detachably fastened to the exterior surface of the display unit to allow a practitioner to manually switch the attached banner to display a different indicium corresponding with a different sanitation status.

In another embodiment, an apparatus, for indicating a sanitation status of a portable surgical cart and/or medical tools stored in or on the portable surgical cart, includes an electronic display, such as a liquid crystal display (LCD) screen. Similarly, in yet one embodiment, an apparatus includes one or more panels of light emitting diodes (LEDs) that are alternatively powered to visually indicate different sanitation statuses. Accordingly, in some embodiment, the apparatus includes a power source, such as a battery, to power an electronic display of the apparatus. The power source may be rechargeable and/or replaceable and may be housed within the apparatus or may be housed within or coupled more directly to portion of the portable surgical cart.

In one embodiment, an apparatus, for indicating a sanitation status of a portable surgical cart and/or medical tools stored in or on the portable surgical cart, is configured to switch between visually indicating different sanitation statuses via a personnel-engageable switch or similar mechanism. According to one implementation, the switch is a physical switch (e.g., a mechanical or electrical switch). In some embodiments, the apparatus includes an electronic communication module (not depicted) that receives actuation commands from a master controller or from a remote user other than the user cleaning or operating the apparatus. In other words, the communication module may allow for a remote controller or a remote user to control the actuation (e.g., switching) of the apparatus. For instance, an administrator monitoring the sterilization procedure for the surgical cart may determine when the sanitation status of the surgical cart has changed and may remotely switch the switchable indicator of the display unit accordingly. The communication may be transmitted via any of various communication protocols, such as hard-wire, wireless, RF, etc., to the communication module of the apparatus to actuate the apparatus and convey the sanitation status of the surgical cart.

In one embodiment, the apparatus of the various embodiments described herein include a locking mechanism that prevents inadvertent or premature switching of the apparatus. For example, regarding a manual switchable indicator, such as the embodiment of the apparatus 620 shown in FIGS. 6A and 6B, the movable cover 628 may be restricted from movement by a mechanical lock (e.g., pad-lock, combination lock, zip-tie, etc.). As another example, such as regarding an apparatus with an electronic switch or electronic display, the locking mechanism may include a user interface or security module that allows only predetermined authorized users to unlock the locking mechanism and switch the apparatus.

Referring to FIGS. 7-9, according to one embodiment, an apparatus 720, for indicating a sanitation status of a portable surgical cart and/or medical tools stored in or on the portable surgical cart, includes a base 730 that defines a channel 732 or recess. Movably retained within the channel 732 is a movable cover 728, which is movable (e.g., slidable as indicated by a directional arrow) within the channel 732 between at least a first position and a second position. The apparatus 720 includes a back panel 752 that interlocks with the base 730 to at least partially define the channel 732. In the illustrated implementation, the back panel 752 interlocks with the base 730 via engagement between locking tabs 750 formed in the back panel 752 and corresponding apertures formed in the base 730. A front side of the back panel 752 includes at least one indicium 726 that visually indicates a sterilization status. Although not shown, the front side of the back panel 752 in some implementations includes two indicia 726 with a first indicium visually indicating a first sterilization status and a second indicium visually indicating a second sterilization status that is different than the first sterilization status. The indicia 726 on the back panel 752 are positioned relative to the channel 732 such that when the movable cover 728 is in the first position, one of the indicia 726 is covered by the movable cover 728, and when the movable cover 728 is in the second position, the other of the indicia 726 is covered by the movable cover 728.

The apparatus 720 further includes a locking feature to releasably lock the movable cover 728 in one of the first position and second position. In this manner, the locking feature helps prevent inadvertent or unauthorized adjustment of the movable cover 728 to avoid confusion as to the actual sanitation status of a portable surgical cart and/or medical tools stored in or on the portable surgical cart. The locking feature of the apparatus 720 includes first upright tabs 734 formed in the base 730 and second upright tabs 729 formed in the movable cover 728. The first upright tabs 734 include apertures 735 and the second upright tabs 729 include apertures 731.

The first upright tabs 734 and the second upright tabs 729 are configured such that when the movable cover 728 is in the first position, one of the first upright tabs 734 of the base 730 is directly adjacent (e.g., abuts) one of the second upright tabs 734 of the movable cover 728, and when the movable cover 728 is in the second position, the other of the first upright tabs 734 is directly adjacent the other of the second upright tabs 729. Moreover, the apertures 735 of the first upright tabs 734 and the apertures 731 of the second upright tabs 729 are positioned such that the respective apertures 735, 731 of a first upright tab 734 and a second upright tab 129, when directly adjacent each other, are at least partially axially aligned. With the apertures 735, 731 at least partially axially aligned, a lock (not shown) can be passed through the apertures 735, 731 and locked to securely fix the movable cover 728 in one of the first or second positions. Then, when the sanitation status of the portable surgical cart and/or medical tools changes, the lock can be unlocked by an authorized personnel (e.g., one possessing a key to the lock), the movable cover 728 can be moved to the opposite position, and the lock can be passed through the apertures 735, 731 of the other first and second upright tabs 734, 729, when directly adjacent each other, and locked to securely fix the movable cover 728 in the other of the first and second positions.

The apparatus 720 also includes attachment features, such as apertures 751 formed in the base 730, that promote attachment of the apparatus 720 to a portable surgical cart. For example, fasteners, securable to the portable surgical cart, may be passed through the apertures 751 and secured to the portable surgical cart to attach the apparatus 720 to the portable surgical cart.

The various components of the apparatus 720 are made of a moldable plastic material. More specifically, the base 730, back panel 752, and movable cover 728 are made from a hardened plastic material formed using a molding technique in some embodiments.

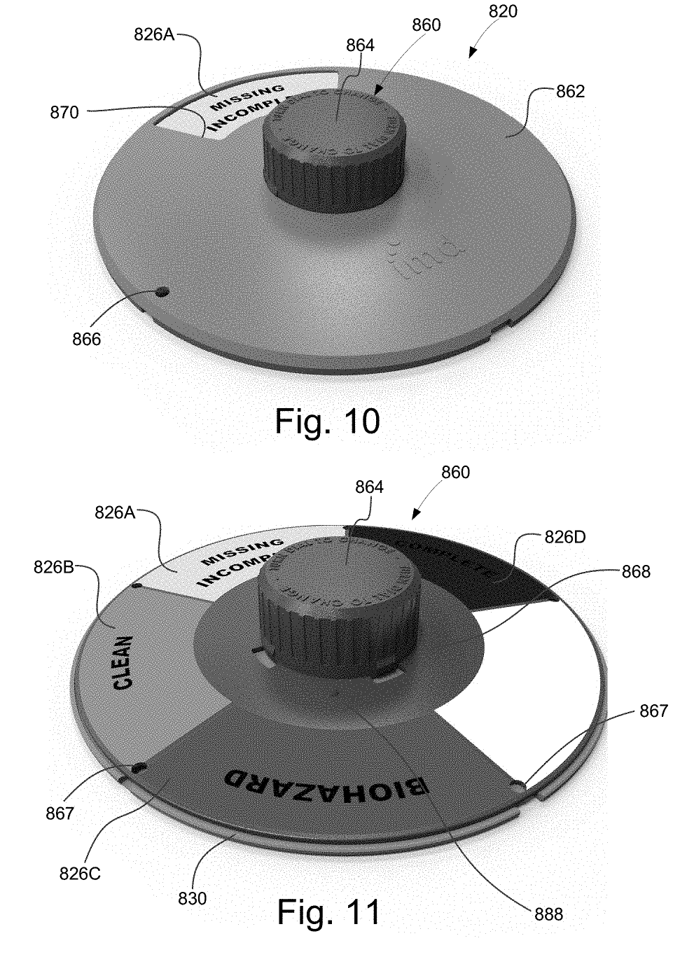

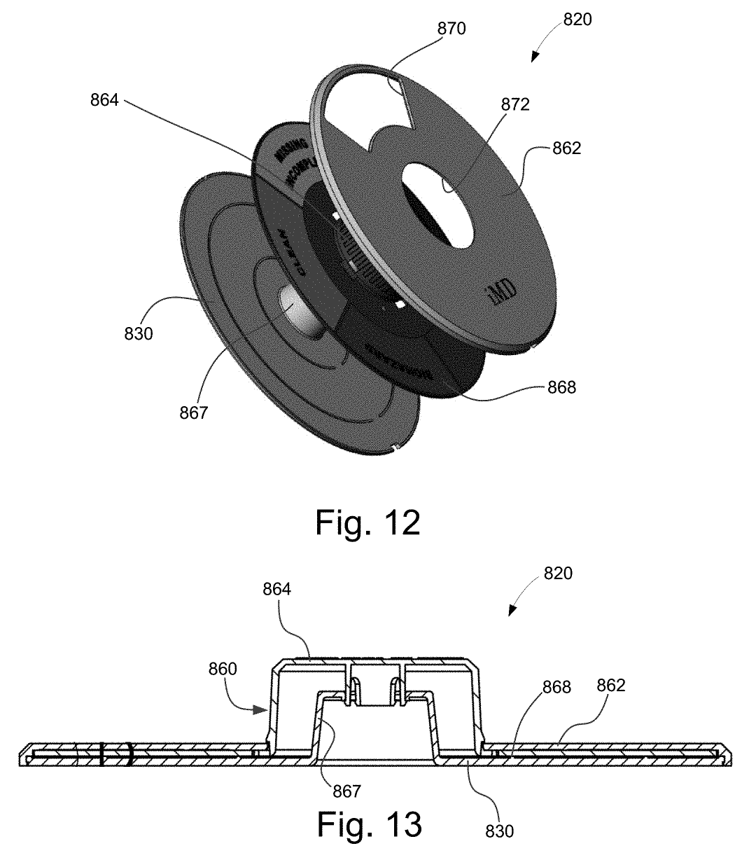

Referring now to FIGS. 10-13, according to one embodiment, an apparatus 720, for indicating a sanitation status of a portable surgical cart and/or medical tools stored in or on the portable surgical cart, includes a base 830 that movably retains a dial 868. In the illustrated embodiment, the base 830 and the dial 868 have a circular, disk-like shape. Engagement between the base 830 and the dial 868 allows the dial 868 to rotate relative to the base 830. For example, as shown in FIGS. 12 and 13, the base 830 includes a spindle 867 and the dial 868 includes a knob 864 that rotationally engages the spindle 867 of the base 830. In one implementation, the knob 864 rotationally engages the dial 868 via a snap-fit arrangement. The knob 864 of the dial 868 includes grip enhancing features, such as ribs, textures, materials, and the like, that promote the grip of the knob 864.

Additionally, the dial 868 includes at least one indicium that visually indicates a sterilization status. In some implementations, the dial 868 includes multiple indicia each visually indicating a different sterilization status. For example, as shown in FIG. 11, the dial 868 includes multiple indicia 826A-D each visually indicating a different sterilization status. With reference to a portable surgical cart, the indicium 826A indicates a sterilized portable surgical cart that does not yet include a complete set of medical tools for a particular surgical procedure. Such a sterilization status would indicate to personnel that more medical tools are needed before the portable surgical cart is ready to be taken to surgery. In contrast, the indicium 826D indicates a sterilized portable surgical that does include a complete set of medical tools for a particular surgical procedure. Such a sterilization status would indicate to personnel that the portable surgical cart is ready to be taken to surgery. The indicium 826C indicates the portable surgical cart is not sterilized or clean. Such a sterilization status would indicate to personnel that the portable surgical cart needs to be sterilized before placing sterilized medical tools on the portable surgical cart in preparation for surgery. Finally, the indicium 826B indicates the portable surgical cart is sterilized or clean and ready to receive sterilized medical tools for surgery. In some implementations, the base 830 and the dial 868 are made from an opaque or non-transparent material, such as plastic. The indicia 826A-D can be printed, adhered, bonded, welded, etc. onto the dial 868.

The apparatus 820 further includes a cover 862 that is non-movably fixed to the base 830 at least partially over the dial 868 such that the indicium 826A-D on the dial 868 is positioned between the cover 862 and the base 830. The portion of the cover 862 over the dial 868 is spaced apart from the base 830 such that, when the cover 862 is fixed to the base 830, the dial 868 is allowed to rotate relative to the cover 862 as well as the base 830. The cover 862 may have a generally circular, disk-like shape similar to the base 830 and the dial 868. The cover 862 is made of an opaque or non-transparent material, such as plastic. Moreover, the cover 862 includes a window 870 sized to correspond with the size and spacing of each of the indicia 826A-D. In other words, the window 870 is positioned on the cover 862 and sized such that only one of the indicia 826A-D is fully visually accessible through the window 870 while all others of the indicia 826A-D are visually obstructed by the cover 862.

The cover 862 also includes a central aperture 872. When the cover 862 is coupled to the base 830, and with the dial 868 interposed between the base 830 and the cover 862, the knob 864 of the dial 868 extends through the central aperture 872 of the cover 862 to be grippable by personnel for rotating the dial 868.

The indicium of the indicia 826A-D that is fully visually accessible through the window 870 of the cover 862 is changeable by rotating the dial 868 relative to the cover 862. For example, if a previously non-sterilized portable surgical cart is sterilized, personnel can rotate the dial 868 in a counter-clockwise direction until the indicium visually accessible through the window 870 changes from the indicium 826C to the indicium 826B. To promote releasable retention of the dial 868 relative to the cover 862, which helps to properly locate indicium within the window 870, the apparatus 820 includes a detect mechanism in some implementations. For example, referring to FIG. 11, the dial 868 includes a detent 888 or protrusion that is configured to releasably engage multiple recesses (not shown) formed in the cover 862 one at a time. The recesses are spaced apart from each other such that when the detent 888 is releasably engaged with one of the recesses, one indicium is fully within the window 870, and when the detent 888 is releasably engaged with another of the recesses, another indicium is fully within the window 870. The detent 888 releases from engagement with a recess when a torque is applied to the dial 868 that overcomes the frictional force between the detent 888 and the recess. In some embodiments, other types of detent mechanisms can be used to releasably retain the dial 868 in multiple positions relative to the cover 862.

In some implementations, the apparatus 820 further includes a locking feature to releasably lock the dial 868 in place with a desired indicium visually accessible through the window 870 of the cover 862. Accordingly, the locking feature helps prevent inadvertent or unauthorized adjustment of the dial 868 relative to the cover 862 to avoid confusion as to the actual sanitation status of a portable surgical cart and/or medical tools stored in or on the portable surgical cart. The locking feature of the apparatus 820 includes an aperture 866 formed in the cover 862, a corresponding aperture (not shown) formed in the base 830, and multiple apertures 867 formed in the dial 868. The aperture 866 of the cover 862 and the aperture of the base 830 are axially aligned, and each aperture 867 of the dial 868 is axially alignable with the apertures of the base 830 and cover 862 when the dial 868 is in a respective one of multiple positions relative to the cover 862, each associated with a different indicium being visually accessible through the window 870. Therefore, when a desired indicium is visually accessible through the window 870, a mechanical lock, such as a padlock, combination lock, zip-tie, and the like, can be passed through the aligned apertures of the base 130, dial 868, and cover 862 and locked to prevent inadvertent or unauthorized rotation of the dial 868 relative to the cover 862 (i.e., inadvertent or unauthorized changing of the indicium visually accessible through the window 870).

Although the medical instrument to which the apparatus is coupled or forms a part is represented in the illustrated embodiments as a portable surgical cart, in other embodiments, instead of a surgical cart, the medical instrument, to which the apparatus is coupled to or forms a part, can be a drip pole, bed, chair, rail, electronic equipment, or any other medical instrument. Accordingly, when used herein, a portable surgical cart can be replaced with any of various other medical instruments without departing from the essence of the subject matter of the present application.

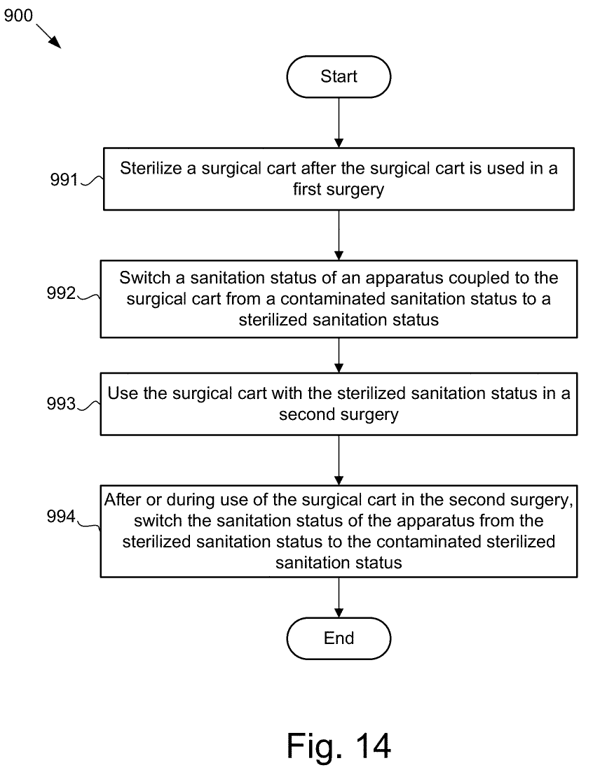

Referring to FIG. 14, and according to one embodiment, a method 900 of indicating the sanitation status of a surgical cart or medical tools includes sterilizing a surgical cart after the cart has been contaminated from a first surgery at 991. As described above, the surgical cart may not have been directly used during surgery. However, the possibility of inadvertent exposure of the surgical cart to a patient's bodily fluid/tissue, or other contaminating objects, may still necessitate sterilization of the surgical cart. After the surgical cart has been sterilized, the method 900 further includes switching a sanitation status of an apparatus coupled to the portable surgical cart from a contaminated sanitation status to a sterilized sanitation status at 992. The portable surgical cart with the apparatus switched to the sterilized sanitation status is then used in a second surgery at 993. During or after the second patient surgery, the method 900 also includes switching the sanitation status of the apparatus from the sterilized sanitation status back to the contaminated sanitation status at 994.

In the above description, certain terms may be used such as “up,” “down,” “upper,” “lower,” “horizontal,” “vertical,” “left,” “right,” and the like. These terms are used, where applicable, to provide some clarity of description when dealing with relative relationships. But, these terms are not intended to imply absolute relationships, positions, and/or orientations. For example, with respect to an object, an “upper” surface can become a “lower” surface simply by turning the object over. Nevertheless, it is still the same object. Further, the terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise. Further, the term “plurality” can be defined as “at least two.”

Additionally, instances in this specification where one element is “coupled” to another element can include direct and indirect coupling. Direct coupling can be defined as one element coupled to and in some contact with another element. Indirect coupling can be defined as coupling between two elements not in direct contact with each other, but having one or more additional elements between the coupled elements. Further, as used herein, securing one element to another element can include direct securing and indirect securing. Additionally, as used herein, “adjacent” does not necessarily denote contact. For example, one element can be adjacent another element without being in contact with that element.

As used herein, the phrase “at least one of”, when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example, without limitation, two of item A, one of item B, and ten of item C; four of item B and seven of item C; or some other suitable combination.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

As used herein, a system, apparatus, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

Many of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of program code may, for instance, comprise one or more physical or logical blocks of computer instructions which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Indeed, a module of program code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network. Where a module or portions of a module are implemented in software, the program code may be stored and/or propagated on in one or more computer readable medium(s).

The present subject matter may be embodied in other specific forms without departing from its spirit or essential characteristics. The described embodiments are to be considered in all respects only as illustrative and not restrictive. All changes which come within the meaning and range of equivalency of the claims are to be embraced within their scope.

What is claimed is:

1. An apparatus for indicating a sanitation status of a medical instrument, the apparatus comprising:
at least one indicium that visually indicates a sanitation status of the medical instrument wherein the sanitation status is one of at least sterilized or contaminated;
wherein the apparatus is configured to be coupled to the medical instrument.

2. The apparatus of claim 1, wherein the apparatus comprises at least two indicium each visually indicating one of at least sterilized or contaminated.

3. The apparatus of claim 2, wherein the apparatus further comprises a cover that is movable relative to at least the two indicium between a first position, covering only one of at least the two indicium, and a second position, covering another of at least the two indicium.

4. The apparatus of claim 3, wherein the cover is slidable between the first position and the second position.

5. The apparatus of claim 4, further comprising a base and a channel defined by the base, and wherein the cover is positioned within and slides along the channel.

6. The apparatus of claim 3, wherein the cover is rotatable between the first position and the second position.

7. The apparatus of claim 6, further comprising a base and a dial, the dial being rotatable relative to the base and including at least the two indicium thereon, and wherein the cover is non-movably coupled to the base such that the dial is interposed between the base and the cover.

8. The apparatus of claim 7, wherein the cover includes a window sized to allow visual access therethrough to only one of at least the two indicium.

9. The apparatus of claim 7, further comprising a detent mechanism that releasably retains the dial in each of the first position and the second position.

10. The apparatus of claim 7, wherein the dial comprises a knob and the cover comprises a central aperture through which the knob of the dial extends.

11. The apparatus of claim 3, further comprising a locking mechanism selectively operable to prevent movement of the cover between the first position and the second position.

12. The apparatus of claim 1, further comprising an electronic display that is switchable between displaying a sanitation status, corresponding with sterilized, and a second sanitation status, corresponding with contaminated.

13. The apparatus of claim 1, further comprising a flip-chart mechanism.

14. The apparatus of claim 1, further comprising at least one liquid drainage port.

15. The apparatus of claim 1, further comprising opposing, angled, or orthogonal sides each including at least the one indicium.

16. A system, comprising:
a portable surgical cart for holding medical tools; and
an apparatus coupled to the portable surgical cart, the apparatus comprising at least one indicium that visually indicates a sanitation status of the portable surgical cart, wherein the sanitation status is one of at least sterilized or contaminated.

17. The system of claim 16, wherein the apparatus is permanently mounted to the portable surgical cart.

18. The system of claim 16, wherein the apparatus is selectively releasably mounted to the portable surgical cart.

19. The system of claim 16, wherein the portable surgical cart comprises a plurality of shelves, and the system further comprises a plurality of apparatuses each coupled to a respective one of the plurality of shelves.

20. A method comprising:
sterilizing a portable surgical cart after the portable surgical cart has been contaminated from a first surgery;
after sterilizing the portable surgical cart, switching a sanitation status of an apparatus that is coupled to the portable surgical cart from a contaminated sanitation status to a sterilized sanitation status;
using the portable surgical cart, with the sterilized sanitation status, in a second surgery; and
after or during use of the surgical cart in the second surgery, switching the sanitation status of the apparatus from the sterilized sanitation status to the contaminated sterilized sanitation status.
